(19) United States
(12) Patent Application Publication
(10) Pub. No.: US 2017/0246445 A1
(43) Pub. Date: Aug. 31, 2017

(54) MULTI-ELECTRODE FLEXIBLE ELECTRIC MASK

(71) Applicant: L'OREAL, Paris (FR)

(72) Inventor: Thi Hong Lien PLANARD-LUONG, Bures sur Yvette (FR)

(21) Appl. No.: 15/500,203

(22) PCT Filed: Jul. 16, 2015
(86) PCT No.: PCT/EP2015/066330

§ 371 Date: Jan. 30, 2017

(30) Foreign Application Priority Data
Jul. 29, 2014 (FR) 1457334

(51) Int. Cl. A61N 1/04; A61N 1/30; A61N 1/32; A61M 37/00; A61M 35/00

(52) U.S. Cl. CPC A61N 1/044; A61M 37/00; A61M 35/00; A61N 1/0476; A61N 1/328; A61N 1/0492; A61N 1/303; A61N 1/325; A61M 2210/0606

(57) ABSTRACT

One subject of the invention is an article for cosmetic treatment of a body area with an electric current, comprising a support (50) comprising an electrode (1) and a counter electrode (2), said electrode and counter electrode being separated from one another by a space comprising an electrically insulating zone (3) made of a polymeric material.

### CROSS REFERENCE TO RELATED APPLICATIONS

This is a national stage application of PCT/EP2015/066330, filed internationally on Jul. 16, 2015, which claims priority to French Application No. 1457334, filed on Jul. 29, 2014, which are incorporated by reference herein in their entireties.

The present invention relates to articles for the cosmetic treatment of human keratin materials with an electric current.

Within the context of the present invention, an “article” is understood to mean a mask, a patch, a pad, a strip or a bandage capable of being applied to human keratin materials.

The expression “cosmetic product” is understood to mean any composition as defined in Council Directive 93/35/EEC of 14 Jun. 1993.

Throughout the text, the expression “comprising a” should be interpreted as being synonymous with “comprising at least one”.

Passive masks for application of cosmetic composition are known. Document U.S. Pat. No. 6702792, for example, describes a cellulose mask impregnated with a facial lotion.

However, the amount of molecules administered via the passive route remains low. In order to improve this aspect, active patches or masks have been developed that use electrodes connected to a power source.

It is thus known to treat human keratin materials using iontophoresis devices (J. Singh, K. S. Bhatia, Topical iontophoretic drug delivery: pathways, principles, factors and skin irritation, Med. Res. Rev., vol. 16, no. 3, 285-296, 1996).

Iontophoresis allows the diffusion of active agents through the skin by virtue of electrical stimulation in a non-invasive manner. The current applied may be adjustable in terms of intensity and direction (anodal or cathodal). The transcutaneous diffusion of the molecules via iontophoresis is based on two principles, namely electrorepulsion and electroosmosis.

Electrorepulsion is the migration of an ionized molecule by repulsion of charges of the same sign. Thus, if a substance is negatively charged, it will diffuse through the skin at the cathode (−).

Electroosmosis is the migration of a molecule, even a non-ionized molecule, by entrainment associated with the flow of water from the anode to the cathode during iontophoresis. The migration is due in particular to the negative charge of the skin. Under the effect of a current, the water or a solvent entrains dissolved substances as it migrates.

The electric current can be applied to the skin by means of an end piece. For large surfaces of the body or of the facial cheek, the end piece may be large. In areas that are more difficult to access, the end piece may take the form of a small head that is easier to bring into contact or to move.

In order to increase the effectiveness of the iontophoresis, it is necessary to develop specific devices which optimize the penetration of the active agents through the skin.

It is already known to use an iontophoretic device provided with a massaging end piece for generating a massage that facilitates this penetration.

By way of illustration, the documents EP 2 430 945 and EP 2 111 889 describe a device for treating the contour of the eye by way of a metal ball connected to the electrode and an integrated cosmetic composition reservoir.

The patent GB 2 372 705 A describes an iontophoretic device having electrodes made of ABS plastic metallized by a corrosion-resistant metal. The reservoir is also connected to the electrode in order to ionize the formulation.

The patent U.S. Pat. No. 6,766,192 B1 describes an iontophoretic device having a ball fixed to a tube of cream.

The professional TMT® device sold by the company Bodyesthetic uses an iontophoretic device provided with a rotary ball made of stainless steel.

The i-beauty gun® device sold by AAMS (Anti-Aging Medical Systems) outputs a current via a probe of the roller type, wherein the deposition of product is regulated on the basis of the impedance measured between an electrode and the skin. With this device, the quantity of product delivered is controlled by an electronic system with a view to reducing the contact impedance with the skin.

It is also known to use iontophoretic masks or patches.

Patents U.S. Pat. No. 6,157,858 and U.S. Pat. No. 7,069,088 describe articles comprising at least one pair of electrodes. The electrodes are placed on a malleable support. They are powered by a generator external to the support located within a housing. Before placing the article on the chosen body area, a cosmetic composition is applied to this area. The article is then placed on the face and the electrodes are powered by electric current. The treatment time varies between 15 minutes and 1 hour.

CN 200951261 describes an article, the active electrode of which covers the whole of the face while the return electrode is attached to another part of the body. Electrical contacts and electric wires connect the electrodes to the power source. The latter is contained in an external housing. The mask is composed of an absorbent layer in contact with the skin, topped with a conductive layer made of rubber. The cosmetic composition is contained in the absorbent fabric.

The article described in patent U.S. Pat. No. 5,443,441 has a base structure similar to that described in CN 200951261, with the difference that the cosmetic composition is not contained in an absorbent fabric but in a reservoir separated from the skin by a semipermeable membrane.

lontophoresis technology is widely used for increasing the penetration of medicaments or of cosmetic active agents. Apart from the chemical factor of the formulation and biological factor of the skin, the penetration performance is dependent on the current intensity and on the application time.

Iontophoresis articles have a major advantage compared to devices. Specifically, they offer the possibility of being able to be applied to the area to be treated for a much longer time than end pieces moving over the face. As the user remains passive during the action of the mask, she accepts it being left on for a long time. On the other hand, as the user must be active, or even exert an effort in order to move the end piece, she will desire a shorter treatment time.

Usually, a user accepts using an iontophoretic device for a care treatment for 2 to 5 minutes, or at most for 10 minutes. In comparison, a user accepts leaving a mask on for a duration of 30 min for example, or even 1 hour.

However, safety regulations (IEC 60-479-1) require that the current passing through the human body be limited to 10 mA DC. For this reason, the acceptable current intensity (in mA/cm²) for a mask is very low. For example, it is of the order of 0.033 mA/cm² at most for a mask having an area of around 300 cm².

However, this current intensity is too low to obtain a significant iontophoretic cosmetic effect on the area treated.

It has already been proposed to increase the amount of active agents penetrating into the skin by electrically insulating the electrodes with an air zone. This is true for the product WrinkleMD Brow Hyaluronic Acid Deep Infusion System® sold by WRINKLEMD. This product comprises a hyaluronic acid iontophoresis patch. It delivers hyaluronic acid to the eye contour area in order to ease swollen and tired eyes and also crow's feet.

There is a need to further increase the amount of active agents penetrating through the skin using an iontophoretic article, while respecting the safety regulations.

There is also a need to increase the effectiveness of the iontophoretic articles, irrespective of the targeted cosmetic treatment.

There is additionally a need to propose an iontophoretic article that enables the uniform application of a cosmetic composition containing an active principle.

The article must additionally be easy to manufacture and to handle.

In particular, there is a need to develop novel articles:

that can be used both with a direct current and with a pulsating current or else with a current consisting of a direct component and a pulsating component,
that ensure the current passes into the skin and not only to the surface thereof,
that prevent the current from penetrating too deeply beneath the skin,
that increase the overall amount of current, while keeping the intensity constant for a wide treatment area.

The invention aims to resolve all or some of the aforementioned needs and also to further improve the articles for cosmetic treatment of human keratin materials with an electric current.

One subject of the invention is an article for cosmetic treatment of a body area with an electric current, comprising a support comprising an electrode and a counter electrode, said electrode and counter electrode being separated from one another by a space comprising an electrically insulating zone made of a polymeric material.

Owing to the electrically insulating zone, the current does not pass over the surface of the skin by taking the shortest path between the electrodes. On the contrary, it is made to bypass the electrically insulating zone by being diverted to inside the skin.

In addition, it is possible to judiciously choose the polymer in order to modify the characteristics of the electrically insulating zone. Thus, the choice of the polymer depends on the insulation desired. The invention thus proposes a wide range of supports having electrical characteristics that can be adapted to the targeted treatment or to the area of the face treated.

The general principle of the invention is the insertion of an electrically insulating zone between the electrode and the counter electrode. The positioning of the electrically insulating zone is optimized in order to control the depth of currents through the skin. It can be compared with the multipolar radiofrequency principle.

The polymeric material is an insulating polymeric material.

In particular, the article according to the invention is a mask for the face.

Advantageously, the electrically insulating zone is designed to prevent the diffusion of a cosmetic composition between the electrode and the counter electrode.

It is a zone that is impermeable to fluids or else a zone that is leaktight with respect to fluids.

According to the invention, the electrically insulating polymeric material advantageously forms an electrically insulating zone, the height of which is greater than or equal to the thickness of the electrodes.

Preferably, the electrically insulating zone defines a wall, the height of which is greater than or equal to the thickness of the electrode and of the counter electrode.

Thus, the effectiveness of the electrically insulating polymeric material is increased.

### GENERAL DEFINITIONS

The article according to the invention may in particular be used with a housing separate from the article, this housing comprising a power supply source. In this case, a new article is connected to the housing at the time of operates in combination with the housing.

The article may also have its power supply source built-in. It then operates autonomously. At the end of each treatment, the user throws away the article with its power supply source.

If the article is a mask, it may have openings for the eyes, the nose and/or the mouth.

The article may be occlusive or non-occlusive, for example being impermeable to steam.

The term “face” should be understood to mean the external area of the anterior part of a human head. The face comprises in particular the chin, the mouth, the lips, the philtrum, the nose, the cheeks, the cheekbones, the eyes, the eyebrows and the forehead.

According to the invention, an “electrode” is understood to be a positively charged electrode (anode) or a negatively charged electrode (cathode). This electrode is generally disposed on the external surface of the article so as to come into direct contact with the keratin materials. However, the electrode may also be inserted into the external wall of the article. In this case, it does not come into contact with the keratin materials directly, but through the cosmetic composition and its support. In general, the electrode is in contact with the area to be treated.

Throughout the text, the term “electrode” means an insulated electrode. An electrode may be in the form of a ball or stud, for example. A “counter electrode” is understood to be a negatively charged electrode (cathode) or a positively charged electrode (anode). The charge of the counter electrode is opposite to that of the electrode.

A “power supply system” is understood to be an electrical assembly that is able to induce a potential difference between the electrodes and the counter electrode.

Electrically Insulating Polymeric Material

The electrically insulating polymeric material forms an electrically insulating zone between the electrode and the counter electrode. This zone completely separates the electrode and the counter electrode so as to electrically insulate them from one another. The electrically insulating polymeric material provides electrical insulation.

The electrically insulating polymeric material may have a visible free surface allowing it to come into direct contact with the skin.

The expression “electrically insulating polymeric material” is understood to mean a material comprising an electrically insulating polymer. This material is also referred to as a dielectric material. It does not allow electric current to pass directly between the electrode and the counter electrode while remaining at the surface of the skin. This material does not conduct electricity.

Geometry of the Electrically Insulating Zone

Preferably, the electrically insulating zone forms a volume defined by:

a height greater than or equal to the thickness of the electrode and of the counter electrode,
a width less than or equal to the distance between the electrode and the counter electrode,
a length less than or equal to the length of the electrode and of the counter electrode.
Advantageously, a geometry of the insulating zone is selected such that:
its width is less than or equal to the distance between the electrode and the counter electrode,
its length is less than or equal to the length of the electrode and of the counter electrode.
This geometry of the electrically insulating zone makes it possible to retain:
zones for absorption of formulation around the electrode and the counter electrode, and
an electrically insulating zone which is not impregnated with the formulations in order to force the current to pass through the skin and not at the surface.

Advantageously, the electrically insulating zone, in particular the wall, does not entirely fill the space between the electrode and the counter electrode.

Parameters of the Electrically Insulating Zone

The electrically insulating zone has an electrical conductivity of zero or almost zero and an infinite resistance (∞Ω).

The electrically insulating zone is also defined by its permittivity and by electric strength.

Advantageously, the electrically insulating zone has an electrical conductivity of less than 10⁻⁶ S.m⁻¹, preferably of less than 10⁻¹² S.m⁻¹.

Permittivity

The permittivity or dielectric constant of an insulator is expressed relative to that of air (equal to that of a vacuum). It is represented by the letter epsilon E and expressed in picofarads/metre.

The vacuum permittivity is equal to:

=ε₀=8.854187·10⁻¹²F·m⁻¹

The absolute permittivity of a material is the product of its relative permittivity (see table below) multiplied by the vacuum permittivity according to the formula: ε=ε₀×ε_{R}

For Teflon it is 18.6 pF/m.

Permittivity and Dielectric Strength of Several Insulators

These values are approximate and may vary markedly as a function of the frequency, of the temperature, of the hygrometry or even of the atmospheric pressure.

Permittivity is also referred to as dielectric constant (symbol εᵣ).

The dielectric strength is in kV/mm

| | | Dielectric strength |
|---|---|---|
| Insulator | Relative permittivity εᵣ | (kV/mm) |
| Dry air | 1 | 4 |
| Rubber | 4 | 15 |
| Silicone rubber | 4.2 | — |
| Kapton ® | | 110 |
| Paraffin | 2.2 | — |
| PVC | 5 | 20 |
| Polyester | 3.3 | — |
| Polyethylene | 2.25 | 18 |
| Polypropylene | 2.2 | — |
| Polystyrene | 2.4 | 24 |
| Polycarbonate | 2.9 | 30 |
| Styroflex | 2.5 | — |
| Teflon | 2.1 | 17 |

Advantageously, the electrically insulating zone is defined by a relative permittivity of strictly greater than 1, preferably greater than 2, and preferably greater than 2.5, when the article is dry.

Advantageously, the electrically insulating zone is defined by an electric strength of strictly greater than 4 kV/mm, preferably greater than 10 kV/mm, and preferably greater than 20 kV/mm.

These values are optimal for obtaining an optimal electrical insulation between the electrode and the counter electrode.

Dimensions

The electrically insulating zone may have a width of between 2 and 10 mm, for example between 2 and 5 mm.

The electrically insulating zone may have a thickness of between 0.2 and 1 mm, and preferably between 0.2 and 0.5 mm.

The electrically insulating zone may be straight and/or curved. Advantageously, the electrically insulating zone may take the form of a straight strip.

Electrically Insulating Polymers

Preferably, the electrically insulating zone comprises a polymer selected from insulating thermoplastic polymers, insulating thermosetting polymers, insulating silicones, insulating thermoplastic elastomers, polyester-based or polyether-based insulating thermoplastic polyurethanes or PVC-based insulating thermoplastic elastomers.

The electrically insulating zone advantageously comprises the following insulating thermoplastic polymers: polyamides (PA), polyolefins or polyalkenes (for example polyethylene PE, polypropylene PP, polymethylpentene PMP, polybutene PB-1, polyethylene terephthalate PET), styrene polymers (for example, polystyrene PS, expandable polystyrene EPS, acrylonitrile butadiene styrene terpolymer ABS), polyacrylics (polymethyl methacrylate PMMA) or else vinyl polymers (for example polyvinyl methyl ether PVME, polyvinyl acetate PVAc, polyvinyl chloride PVC).

Alternatively, the electrically insulating zone advantageously comprises the following insulating thermosetting polymers: polyurethanes (PU) originating from the reaction of an isocyanate with hydroxylated groups in order to form a flexible open-cell foam suitable for contact with the skin.

Alternatively, inorganic polymers, the main chain of which does not comprise carbon atoms, are advantageously used as constituent material of the electrically insulating zone, especially polysiloxanes or silicones in common parlance. Some examples of the silicones used are polydimethylsiloxane (PDMS), silicone rubber comprising methyl and phenyl groups (PMQ), silicone rubber comprising methyl, phenyl and vinyl groups (PVMQ) or else silicone rubber comprising methyl and vinyl groups (VMQ).

The material constituting the separation zone may also advantageously be selected from insulating thermoplastic elastomers TPE, such as thermoplastic styrene elastomers (for example butadiene and styrene copolymers SBS and ethylene, butylene and styrene copolymers SEBS), thermoplastic polyurethanes TPU based on polyester (AU) or based on polyether (EU), and thermoplastic elastomers based on PVC (TPE/PVC).

The material constituting the electrically insulating zone may also be an ink, such as that described in WO 2009150972, EP-A-0 016 498 or EP 0 168 849.

The material constituting the electrically insulating zone may be deposited on the article by pressurized jet followed by drying and evaporation of the solvents or by the screen printing process. The material constituting the electrically insulating zone may be chemically impregnated on the article.

Electric Power Source

The article may comprise, within it, an electric power source.

The incorporation of the electric power source of the mask may make it possible to do away with electric wires connecting the mask to an external source. The ease of use of the mask is then increased and it becomes easier for the user to use the mask while moving about or in the bath.

Advantageously, the article comprises an electric power source, located on the support.

The electric power source may be attached to the article in a removable or non-removable manner.

The electric power source may, for example, be incorporated into the mask during its manufacture. The power source may be sandwiched between two outer layers of the article.

It is also possible for the electric power source to be installed in the article just before the treatment. It may then be offered to the user separately from the mask.

The electric power source may comprise a DC voltage source. As a variant, the electric power source may comprise an electronic circuit for varying the amplitude of the voltage generated over time. This electronic circuit may, for example, be a chopper or a constant current generator.

The electric power source may, for example, be connected to the electrodes without a cutout. In this case, the electrical resistance between the electrodes may be high enough so that the electric power source does not discharge when the article is not in operation.

It is also possible to start the electric power source by pushing on a portion of the mask in order to bring said electric power source into contact with a conductive path.

Electrical Parameters

The electrical power source may comprise any non-rechargeable battery or any storage battery. The potential difference between the electrodes is for example between 1.2 V and 24 V, preferably between 1.2 and 10 V. If appropriate, the passage of the current can create spot heating.

At an equivalent current density, the article can in particular deliver a current density, at the skin, of preferably less than or equal to 0.500 mA/cm², for example between 0.01 mA/cm² and 0.500 mA/cm², for example between 0.01 mA/cm² and 0.10 mA/cm².

The Various Types of Currents

It is possible to use a direct current, an alternating current or a pulsating current to power the article according to the invention.

It is possible to cut the mask into several compartments. Each compartment comprises an electrode.

Advantageously, the article comprises at least two compartments each comprising an electrode and a counter electrode and the article is powered by a sequential current, in particular a sequential current for which the base element is a direct, alternating or pulsed current.

The sequential current is obtained by installing a switch. A current switching device, in other words a switch, is installed with the generator. This switch makes it possible to change the active state of the current sent to each compartment. Specifically, the current is sent in sequences to each compartment. Each sequence lasts between 1 second and 1 minute, preferably between 1 second and 10 seconds.

The article may also be powered by a sequential current and a continuous current of low intensity.

Advantageously, the generator is designed so that the user can change the polarity of the current.

Thus, the article enables, at will, extraction of impurities from the body area, care of the body area or making up of the body area.

Electrodes

At least one electrode may comprise, for example:

a composite material (plastics material loaded with carbon microfibres),

a conductive woven fabric,

a conductive nonwoven fabric,

a polymeric material rendered conductive,

a fibrous material,

conductive polymeric fibres, for example as described in the publication CN101532190,

carbon fibres, for example as described in the publication JP2009179915,

silicones rendered conductive by the addition of conductive fillers such as silver, copper or carbon. Such silicones are supplied, for example, by the companies Saint Gobain, Plastics Performance and Aquitaine Caoutchouc 2000,

conductive metallic fabrics, supplied for example by the companies Utexbel and Cousin Biotech,

carbon-loaded vinyl, supplied for example by the companies Copema and Rexam,

electrosurgical plates, supplied for example by the companies Copema and 3M,

intrinsically conducting polymers, supplied for example by the company Paniplast.

The “active surface of an electrode” is understood to mean the surface of an electrode in contact with the body area, when the article is in place on said body area.

Advantageously, the electrode and the counter electrode are flexible. Thus, they follow the movements of the article and are simple to place on the face. They adopt the curves of the face better.

Preferred Electrodes and Counter Electrode

Advantageously, the electrode and the counter electrode form an array that is spread out over the support. Thus, it is possible to treat a maximum and uniform surface area of the face.

Preferably, the array comprising a comb of electrodes arranged along a first direction and a comb of counter electrodes arranged along a second direction opposite the first direction.

The electrodes and the counter electrodes may be parallel to one another.

The comb of electrodes and the comb of counter electrodes may be arranged so that the electrodes and the counter electrodes are interlaced in pairs. In other words, between two adjacent electrodes there is a counter electrode. There is a space on either side of the counter electrode. Conversely, an electrode may be between two counter electrodes. In this case there may be a space on each side of the electrode so as not to have any contact between the electrode and the counter electrodes.

An array of electrodes created from such an arrangement is referred to as an “interdigitated array of electrodes”.

Cosmetic Composition

The cosmetic composition may be deposited directly on the area to be treated by the user.

The cosmetic composition may also be impregnated or dispersed in the article.

The active principle is, preferably, charged. The term “charged” is understood to mean any active principle present at least partially in ionic form, the ions of which have an either positive or negative net charge, capable of ensuring their mobility within the composition under the effect of an electric field.

Thus, the active agent is directly subjected to the attraction or repulsion of the electrodes.

Advantageously, the cosmetic composition is selected from care, washing, purifying, exfoliating, desquamating, massage, slimming, makeup, makeup removal, cleansing or bleaching compositions.

More advantageously, the cosmetic composition is in the form of an aqueous solution, an oil, an emulsion, a powder or a gel.

Irrespective of the embodiments considered, the article may exert an action on the skin via iontophoresis and/or electroosmosis.

The composition may comprise a composition for activating an unactivated active principle present within the article, for example in freeze-dried form. In this case, the composition may be free of a charged active principle. The composition may comprise a solvent having positively and negatively charged species, for example an ionic aqueous solution or an aqueous solution of deionized water or else a solution of NaCl.

It is also possible for the user to apply an activation composition, for example a solvent, to the article. For example, the user may apply running water, when no water is provided within one and the same packaging with the article.

In order to bring the article and the activation composition into contact, the user may pour the composition onto the article. The latter is, for example, present in a pouch or tray enabling the composition to be poured thereon. As a variant, the user may apply the composition to the skin, then apply the article on top.

Electrochemical Reaction

Generally, when it is sought to administer an active principle using the mask according to the invention, said active principle has the same polarity as the electrode. For example, the compounds containing active principles of positive polarity/charge, such as vitamin A, tocopheryl acetate or other active principles of positive charge/polarity, may be combined with an electrode of positive polarity.

The compounds containing active principles of negative polarity/charge such as retinyl palmitate, tocopherol, mandelic acid or ascorbic acid may, for their part, be combined with an electrode of negative polarity.

The Support

Dimensions and structure

The support may, in the unfolded state, have a surface area of between 5 cm² and 500 cm², for example between 50 cm² and 300 cm².

The support may, in the unfolded state, have a thickness of between 0.5 cm and 1 cm, and preferably between 0.1 cm and 0.5 cm.

The support may be configured in order to produce a sound and/or light signal that makes it possible, for example, to alert the user that it has been left on the skin for the required time.

After use, the article may be thrown away in its entirety.

Support Material

Advantageously, the support comprises a nonwoven material. The support is thus more flexible. It adapts better to the contours of the chosen area.

Nonwoven

Within the meaning of the present invention, the term “nonwoven” is understood to mean a substrate comprising fibres, wherein the individual fibres or the filaments are arranged in a disordered manner in a structure in the form of a sheet. The fibres of the nonwoven are generally bonded together, either under the effect of a mechanical action, or under the effect of a thermal action, or by addition of a binder.

Such a nonwoven is, for example, defined by standard ISO 9092 as a web or sheet of directionally or randomly orientated fibres, bonded by friction and/or cohesion and/or adhesion, excluding paper and products obtained by weaving, knitting or stitching incorporating binding yarns or filaments.

Advantageously, the weight percentage of hot-melt fibres contained in the nonwoven is greater than 0.5% and is less than or equal to 100%, advantageously between 5% and 80%.

The hot-melt fibres are for example polyolefin fibres, such as polyethylene (PE), polypropylene (PP) or polyethylene terephthalate (PET) fibres or acrylic fibres such as polymethyl methacrylate (PMMA) fibres, polyurethane fibres or fibres of the following thermoplastics: polyvinyl chloride (PVC), styrene polymers (for example polystyrene PS, expandable polystyrene EPS, acrylonitrile-butadiene-styrene terpolymer ABS, styrene-acrylonitrile copolymer SAN, styrene-butadiene copolymer SB), polyamides (PA), polycarbonates (PC), saturated polyesters (for example polyethylene terephthalate glycol PET, polybutylene terephthalate glycol PBT), polyacetals (for example polyoxymethylene POM, trioxane-ethylene oxide copolymer), to polyvinyl alcohol (PVA), or else fluoropolymers (for example polytetrafluoroethylene PTFE, polyvinylidene fluoride PVDF, polychlorotrifluoroethylene PCTFE).

The other fibres constituting the nonwoven are for example synthetic fibres derived from petroleum derivatives, natural fibres derived from plants or animals, and/or modified natural fibres, derived for example from treatment or regeneration processes in order to form fibres.

The nonwoven may be formed from one or more consolidated fibre webs.

Conductivity of Nonwoven

The conductivity of a nonwoven array depends on the type, the amount, the orientation and the distribution of the conductive fibres incorporated. A nonwoven may have a resistance of less than 1500 ohms/square, or else less than 100 ohms/square, for example less than 10 ohms/square.

The conductivity of a sample is calculated as being the quotient of the resistance of the sample, expressed in ohms, divided by the ratio of the length to the width of the sample. The resulting resistance of the sample is expressed in ohms per square. More specifically, the resistance measurement may be in accordance with the “Method for Determining the Resistivity of a Printed Conductive Material” ASTM F1896-98 as described in patent application

The process for creating the electrically insulating zone comprises the steps consisting in:

providing a woven or nonwoven support,

laying the support flat and optionally stretching it in order to have a flat surface,

positioning the electrode and the counter electrode on the support,

defining the size and the geometry of the electrically insulating zone,

optionally, marking the electrically insulating zone with a boundary, in particular with a mould,

choosing an electrically insulating polymer, the melting point of which is markedly below the melting point of the constituent material of the support,

depositing the electrically insulating polymer in the molten state at the boundary,

allowing the electrically insulating polymer to cure by cooling down to ambient temperature,

optionally, removing the mould,

optionally, turning the support over and carrying out the same steps again on the reverse side of the support.

Another implementation process is screen printing of the insulating polymer anchor on the support.

The steps described above are part of a process of “double printing” of a polymer on a support. This process advantageously makes it possible to have a deep and uniform diffusion of the material of the electrically insulating zone in the support. An electrically insulating barrier is formed between the electrode and the counter electrode, preventing the migration of the active components out of the desired zone.

The process for printing conductive or insulating circuits on the flexible supports of very thin film or nonwoven type is generally used for producing the articles. Use is usually made of conductive ink for the electrodes and of insulating polymer or silicone ink for the electrically insulating zone.

The electrically insulating zone, made of one of the electrically insulating materials cited above, adheres perfectly to the body area, in particular to the skin, at its contact surface. This adhesion ensures the absence of moisture or air between the contact surface with the skin. Furthermore, once the active components located between the electrode or the counter electrode and the electrically insulating zone are deposited at the surface of the skin, these components do not intermingle in the neighbouring deposition zone. The active components therefore remain concentrated on the body area for which they are intended. This concentration improves the penetration of the components into the skin via a two-fold mechanism: occlusion and iontophoresis.

The invention also relates to a cosmetic care or makeup method to comprising the steps consisting in:

installing an article as defined above on a body area, in particular on the face,

circulating an electric current within said article thus installed.

According to another of its aspects, one subject of the invention is a is method for extracting compounds, in particular cosmetic cleansing compounds, from the skin, for example from the epidermis, comprising at least the successive steps consisting in:

installing an article as defined above on the face; and

circulating an electric current within said mask thus installed in order to enable the extraction of at least one compound present within the skin.

This method may be a method for cleansing the skin, especially that of the face. In this case, the first electrode may cover, at least partly, the nose and/or the forehead and/or the cheekbones. The electric current may promote the migration of one or more species from the skin to the mask, it being intended for these species to be eliminated or to help to convey away, in their migration, one or more compounds to be eliminated.
The compounds extracted from the skin may, for example, be impurities, ions, peptides, proteins, amino acids, polysaccharides, residues of makeup or deposits of dust.
The compounds extracted from the skin may also be residues of a composition previously applied, for example by a mask as defined above.
The compounds extracted from the skin may or may not be charged.
When these compounds are charged, they may have a polarity opposite to that of the electrode to which they are attracted.

### DESCRIPTION OF THE FIGURES

The invention may be better understood from reading the following description of nonlimiting illustrative embodiments thereof and from examining the appended drawing, in which:

FIG. 1 is a schematic front view of an exemplary embodiment of an electrode array according to the invention,

FIG. 2 is a schematic front view of an exemplary embodiment of an article including electrode arrays according to the invention,

FIG. 3 represents a cross-sectional view of a portion of the electrode array of FIG. 1 according to the invention.

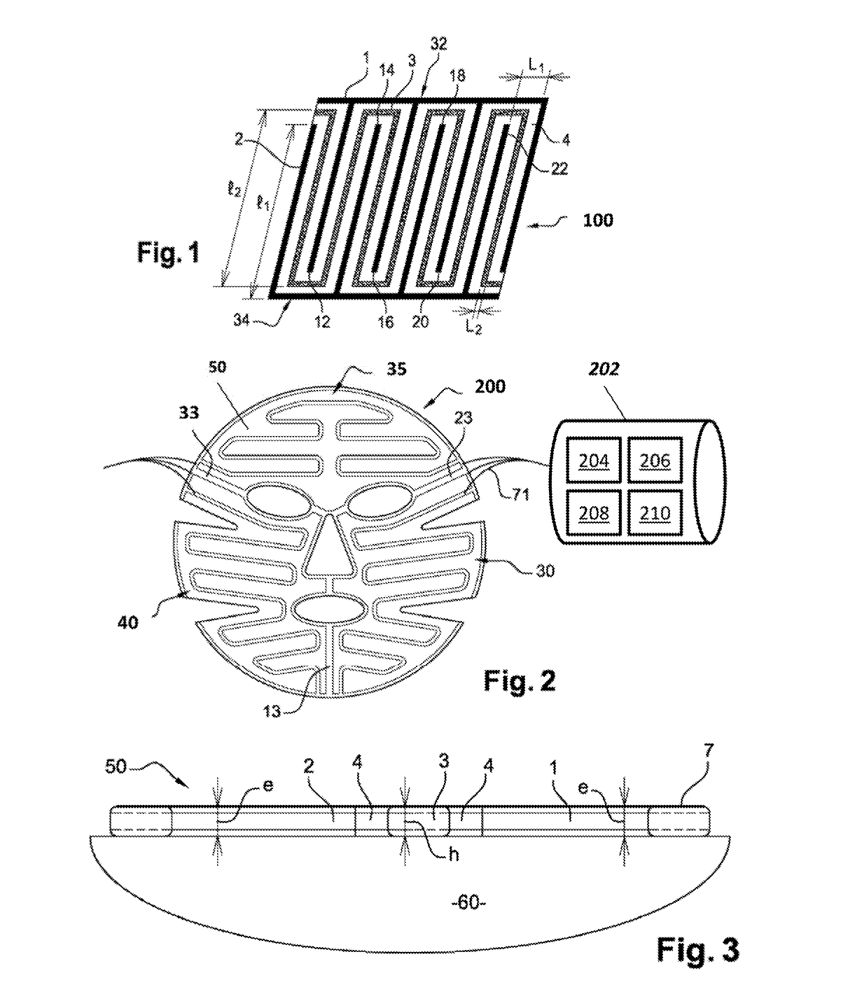

In FIGS. 1 to 3, the actual relative proportions of the various elements have not always been respected, for the sake of clarity.

Referring to FIGS. 1 and 3, the electrode array 100 comprises an electrode 1 and a counter electrode 2, said electrode 1 and counter electrode 2 being separated from one another by an electrically insulating zone 3 made of polymeric material.

The polymeric material may be a material selected from those listed in the table below.

| Polymeric materials | Commercial name | Producer |
|---|---|---|
| HDPE | DOW ™ | DOW |
| LDPE | Elite ® | DOW |
| Polypropylene | Velvex ™ | Styron |
| Polybutadiene | Arinte ® | DSM |
| PET | Rynite ® | DuPont |
| Polystyrene | Styrosolution ®PS | Styrosolution |
| ABS - acrylonitrile | Sicoflex ® | Ravago |
| butadiene styrene | | |
| terpolymer | | |
| PMMA | Altuglas ® PMMA | Altuglas |
| | | International-Arkema |
| PVC | S-58-02 | Shin Etsu |
| Silicones | Tego ®RC Silicones | Evonik Industries |
| TPE | Enflex ® | Ravago |
| TPU | Irogran ® | Huntsman |
| Thermoplastic | Hytrel ® | DuPont |
| polyester elastomer | | |

The electrically insulating zone 3 does not entirely fill the space 4 between the electrode 1 and the counter electrode 3.

The electrically insulating zone forms a wall defined by:

a height h equal to the thickness e of the electrode and of the counter electrode,

a width L₂ of less than the distance L₁ between the electrode and the counter electrode.

a length l₂ equal to the length l₁ of the electrode and of the counter electrode.

If the support 50 is impregnated with a cosmetic composition, this composition may be accomodated between the electrically insulating zone 3 and the electrode 1 or the counter electrode 2.

If the cosmetic composition comprises positively charged active agents, the portion of the support 50 in contact with the positive electrode 1 pushes the active agent towards the skin. Then by reversing the polarity, the other portion of the support 50 is positively charged and so on and so forth. For a uniformity of treatment, it is possible to reverse the current from time to time (every minute for example). This type of support 50 may be used in order to form a patch. It may then be manufactured on a reel then cut to size.

The electrode 1 and the counter electrode 2 are spread out to form the array 100.

In the electrode array 100, the electrode 1 forms a first comb 32 comprising teeth lined up along a first direction. Three of the teeth of the comb 32 are denoted by 12, 16, 20. In the array 100 the counter electrode 2 forms a second comb 34 comprising teeth lined up along a second direction opposite to the first direction. Three of the teeth of the counter electrode are referenced by 14, 18, 22. The first comb 32 and the second comb 34 are arranged so that the teeth 12, 16, 20 of the first comb 32 and the teeth 14, 18, 22 of the second comb are alternately interlaced in pairs. The interlacing is carried out following a periodic array pitch.

The article represented in FIG. 2 is a mask 200 to be applied to the skin of the face.

The mask 200 comprises a support 50.

The mask 200 comprises three compartments 30, 35, and 40.

Each compartment comprises an electrode array 100 including an electrode 1 and a counter electrode 2 separated by an electrically insulating zone 3.

This electrically insulating zone 3 is additionally a leaktight and hermetic zone. It blocks the passage of the cosmetic composition.

The compartments 30, 35, and 40 are separated from one another by additional electrically insulating zones 23, 33, and 13.

This type of article may be used for a large treatment area. It is possible to program a current intensity adapted to each area of the face. These areas have neither the same sensitivity, nor the same tolerance.

The mask 200 is connected to a handle 202 by cables 71.

Found in the handle 202 are a current generator 204, control buttons 206, a battery 208, and a programmable switch 210. The latter makes it possible to power the cables 71 in a sequential or synchronised manner as necessary. The maximum total current may range up to 5 mA.

FIG. 3 represents a cross-section of a portion of the electrode array 100 of FIG. 1, which may be included in the mask 200. The array 100 includes an electrode 1 and a counter electrode 2. This mask 200 is applied to the skin 60. The electrode and the counter electrode are separated by an electrically insulating zone 3. This zone is created by double printing of an insulating barrier ink. Thus, the ink penetrates deeply into the fibres of the support 50. The barrier ink creates an electrically insulating zone 3. The electrically insulating zone 3 does not completely fill the spaces 4 between the electrode 1 and the counter electrode 2. These spaces 4 may be impregnated with a cosmetic composition.

In this case, the barrier ink prevents the conductive cosmetic composition from migrating into the thickness of the support 50.

An electrically insulating hermetic film 7 adheres to the support at the barrier ink. It is chosen so that the flexibility of the whole of the mask 200 is preserved. With the barrier ink, it delimits an electrically insulating zone 3 which moreover prevents the cosmetic composition from migrating from the electrode 1 to the counter electrode 2 by bypassing the barrier ink.

The electrically insulating zone 3 forms a wall defined by a height (h) equal to the thickness (e) of the electrode 1 and of the counter electrode 2.

Furthermore, the electrically insulating zone 3 generates good contact with the skin 60 by adhesion. A good electrical insulation between the electrodes 1 and 2 is ensured.

This method of manufacturing the mask as multiple layers also ensures a long-lasting moisture of the mask throughout the treatment time. It also improves the penetration performance of the cosmetic active agents through the two-fold mechanism of occlusion and iontophoresis.

The path of the printing of the barrier ink does not need to be very wide, from 2 mm to 5 mm is sufficient to create optimal insulation.

In order to use the mask presented in FIG. 3, the procedure is performed as follows.

The mask is placed on the face.

The user triggers the operation thereof via a control button.

The treatment time varies between 15 minutes and 1 hour, the limiting factor possibly being the battery operating time.

Once the treatment is finished, the mask is not reused but it could be reused some other way.

The invention is not limited to the examples that have just been described.

The implementation characteristics of the examples illustrated may be combined together within variants that are not illustrated. The structure of the electrodes and the surface area that they occupy in each compartment may in particular be different. The nature of the support, and the number and the size of the compartments may be different. The same is true for the nature of the chemical compositions and the polarity of the electrodes.

1-13. (canceled)

14. An article for cosmetic treatment of a body area with an electric current, comprising:
a support; and
an electrode array disposed on the support, the electrode array comprising:
an electrode disposed on the support;
a counter electrode disposed on the support adjacent to the electrode; and
an electrically insulating polymeric material disposed between the electrode and the counter electrode and configured to form an electrically insulating zone between the electrode and the counter electrode.

15. The article of claim 14, wherein:
the electrode comprises a comb electrode;
the counter electrode comprises a counter comb electrode; and
teeth of the comb electrode are interlaced with teeth of the counter comb electrode.

16. The article of claim 14, wherein the polymeric material is configured to prevent diffusion of a cosmetic composition through the support, between the electrode and the counter electrode.

17. The article of claim 14, wherein the polymeric material defines a wall having a height that is at least equal to a thickness of the electrode and/or the couther electrode, taken in a direction perpendicular to a plane of the support.

18. The article of claim 14, wherein the polymeric material does not completely fill a space between the electrode and the counter electrode.

19. The article of claim 18, further comprising a cosmetic composition disposed in the space between the electrode and counter electrode.

20. The article of claim 14, wherein polymeric material has an electrical conductivity of less than about 10⁻⁶ S. m⁻¹.

21. The article of claim 14, wherein the polymeric material has a relative permittivity of greater than about 1 when the article is dry.

22. The article of claim 14, wherein the polymeric material is selected from insulating thermoplastic polymers, insulating thermosetting polymers, insulating silicones, insulating thermoplastic elastomers, polyester-based or polyether-based insulating thermoplastic polyurethanes, and/or PVC-based insulating thermoplastic elastomers.

23. The article of claim 14, wherein:
the article is divided into compartments;
each of the compartment comprises an electrode array; and
each of the electrode arrays is independently connected to a sequential current source.

24. The article of claim 23, further comprising at least one insulating zone configured to electrically insulate the compartments from one another.

25. The article of claim 23, wherein each of the electrode arrays is arranged in a serpentine pattern in each compartment.

26. The article of claim 14, further comprising a generator configured to apply a current between the electrode and counter electrode.

27. The articles of claim 26, wherein the generator is configured to selectively change a polarity of the current.

28. A cosmetic care or makeup method comprising:
applying an article to skin, the article comprising:
a support; and
an electrode array disposed on the support, the electrode array comprising:
an electrode disposed on the support;
a counter electrode disposed on the support adjacent to the electrode; and
an electrically insulating polymeric material disposed between the electrode and the counter electrode and configured to form an electrically insulating zone between the electrode and the counter electrode; and
circulating an electric current though the electrode array while the article is applied to the skin to increase the penetration of a cosmetic active agent into the skin.

29. The method of claim 28, further comprising loading the support with a cosmetic prior to applying the article to the skin.

30. A method of extracting impurities from the skin, the method comprising:
applying an article to skin, the article comprising:
a support; and
an electrode array disposed on the support, the electrode array comprising:
an electrode disposed on the support;
a counter electrode disposed on the support adjacent to the electrode; and
an electrically insulating polymeric material disposed between the electrode and the counter electrode and configured to form an electrically insulating zone between the electrode and the counter electrode; and
circulating an electric current though the electrode array while the article is applied to the skin to remove impurities from the skin
